(19) United States
(12) Patent Application Publication
(10) Pub. No.: US 2012/0156328 A1
(43) Pub. Date: Jun. 21, 2012

(54) METHODS FOR PREPARING CAKES USING PHOSPHOLIPASES AND CAKE BATTER AND CAKE MIX COMPOSITIONS COMPRISING PHOSPHOLIPASES

(75) Inventors: Leonardo De Maria, Frederiksberg (DK); Jesper Vind, Vaerloese (DK); Morten Tovborg Jensen, Vaerloese (DK); Ingrid Van Haesendonck, Mechelen (BE); Goedele Van der Biest, Zellik (BE)

(73) Assignee: PURATOS N.V., Groot-Bijgaarden (BE)

(21) Appl. No.: 13/376,762

(22) PCT Filed: Jun. 8, 2010
(86) PCT No.: PCT/EP10/58034

§ 371 Date: Feb. 28, 2012

(30) Foreign Application Priority Data
Jun. 10, 2009 (EP) 09162448.6

(51) Int. Cl. C12N 9/18; A21D 10/04

(52) U.S. Cl. 426/47; 435/197; 426/61

(57) ABSTRACT

The present invention relates to methods for preparing a cake using phospholipase variants and cake and cake mix compositions comprising same.

### REFERENCE TO A SEQUENCE LISTING

This application contains a Sequence Listing in computer readable form. The computer readable form is incorporated herein by reference.

### FIELD OF THE INVENTION

The present invention relates to methods for preparing cakes using phospholipase variants.

### BACKGROUND OF THE INVENTION

WO 98/26057 discloses a lipase/phospholipase from Fusarium oxysporum and its use in baking. Soragni et al., 2001, EMBO J. 20: 5079-5090 discloses a phospholipase (TbSP1) from Tuber borchii and the nucleotide sequence of a cDNA of a gene encoding it. WO 2004/097012 discloses a phospholipase from Fusarium venentatum and nucleic acid sequence of a gene encoding it. WO 00/32758 discloses lipolytic enzyme variants having phospholipase and galactolipase activity and their use in baking. WO 2008/025674 discloses the use of phospholipases to reduce the amount of eggs used in cakes.

### SUMMARY OF THE INVENTION

The present invention is directed to methods for preparing cakes using a phospholipase, wherein the phospholipase comprises an alteration at one or more positions corresponding to positions 1, 6, 30, 31, 33, 38, 39, 42, 43, 44, 45, 47, 52, 59, 61, 64, 65, 77, 84, 102, 106, 110, 116, 119 or 120 of the mature polypeptide of SEQ ID NO: 2 and/or an amino acid extension at the N and/or C terminus of the phospholipase. In an embodiment, the present invention provides a method for preparing a cake comprising:

a) preparing a cake batter comprising egg yolk lecithin and a phospholipase of the present invention:

b) baking the cake batter to make a cake.

The phospholipase may be applied in the preparation of a cake in any suitable manner, including, for example, by direct addition of the phospholipase to a cake batter, by addition of a phospholipase to ingredients used to prepare a cake batter (prior to formation of the cake batter), or by treatment of cake ingredients, such as, egg yolk, egg yolk lecithin or a non-egg protein, separate from the cake batter, followed by inclusion of the treated ingredients in a cake batter). The phospholipase may also be applied in the form of a dry mix comprising one or more cake batter ingredients.

The invention also provides a method for preparing a cake using a reduced amount of egg or egg protein (e.g., egg yolk lecithin) as compared to the amount of egg or egg protein used in conventional cake recipes. Accordingly, in another embodiment, the method comprises contacting a cake batter with a phospholipase of the present invention, wherein the cake batter contains 0.3-1.5% by weight of egg lecithin or 5-25% by weight of whole egg.

In another embodiment, the method further comprises contacting the cake batter with a combination of a non-egg phospholipid or protein source and a phospholipase of the present invention.

In another embodiment, the method further comprises contacting the cake batter with a combination of a non-egg phospholipid or protein source and a phospholipase of the present invention, while also using a reduced amount of egg or egg protein in the cake recipe. In yet another embodiment, the method further comprises contacting the cake batter with a combination of a non-egg phospholipid or protein source and a phospholipase of the present invention, wherein the cake batter contains 0.3-1.5% by weight of egg lecithin or 5-25% by weight of whole egg.

The present invention is also directed to cake batter compositions comprising a phospholipase of the present invention and to a dry mix of compositions comprising a phospholipase of the present invention and one or more cake batter ingredients.

### DETAILED DESCRIPTION OF THE INVENTION

### Definitions

Phospholipase activity: The term “phospholipase activity” is defined herein as enzymatic activity that catalyzes the release of fatty acyl groups from a phospholipid. A phospholipase may also catalyze the release of fatty acyl groups from other lipids. For purposes of the present invention, phospholipase activity may be determined in the LEU assay by hydrolyzing soy lecithin (L-α-phosphatidyl-choline from soybean Sigma P5638). The reaction mixture of 20 g/L lecithin, 3.2 mM sodium deoxycholate, 6.4 mM calcium chloride is kept at pH 8.0 during the reaction (2 minutes) at 40° C. Phospholipase activity is expressed as the rate of titrant consumption (0.1 M NaOH) necessary for keeping constant pH, relative to a standard, during neutralization of the liberated fatty acid.

Variant: The term “variant” is defined herein as a polypeptide having phospholipase activity comprising an alteration (substitution, insertion, and/or deletion or N or C terminal extension) of one or more (several) amino acid residues at one or more (several) specific positions. The altered polynucleotide is obtained through human intervention by modification of the polynucleotide sequence, e.g., the polynucleotide sequence disclosed in SEQ ID NO: 1; or a homologous sequence thereof. The variant may also be prepared by gene synthesis or any other method suitable for obtaining a nucleic acid sequence of interest encoding the variant phospholipase.

Wild-Type Enzyme: The term “wild-type” denotes a phospholipase expressed by a naturally occurring microorganism, such as a bacterial, yeast, or filamentous fungus found in nature, and which nucleic acid sequence encoding the wild-type enzyme has not been altered by human intervention.

Parent Enzyme: The term “parent” as used herein means a phospholipase to which a modification, e.g., substitution(s), insertion(s), deletion(s), and/or truncation(s), is made to produce the enzyme variants of the present invention. This term also refers to the polypeptide with which a variant is compared and aligned. The parent may be a naturally occurring (wild-type) polypeptide or a variant. For instance, the parent polypeptide may be a variant of a naturally occurring polypeptide which has been modified or altered in the amino acid sequence. A parent may also be an allelic variant, which is a polypeptide encoded by any of two or more alternative forms of a gene occupying the same chromosomal locus.

Isolated: The term “isolated,” as in “isolated polypeptide” or “isolated phospholipase variant” or “isolated polynucleotide,” as used herein refers to a variant or a polypeptide that is isolated from a source (microorganism). In one aspect, the variant or polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE. In one aspect, the isolated polynucleotide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, and even most preferably at least 95% pure, as determined by agarose electrophoresis.

Substantially pure: The term “substantially pure” or denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure variant or polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The variant phospholipases of the present invention are preferably in a substantially pure form. This can be accomplished, for example, by preparing the variant phospholipase by well-known recombinant methods or by classical purification methods.

The term “substantially pure polynucleotide” as used herein refers to a polynucleotide preparation free of other extraneous or unwanted nucleotides and in a form suitable for use within genetically engineered polypeptide production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form, i.e., that the polynucleotide preparation is essentially free of other polynucleotide material with which it is natively or recombinantly associated. The polynucleotides may be of genomic, cDNA, RNA, semisynthetic, synthetic origin, or any combinations thereof.

Mature polypeptide: The term “mature polypeptide” is defined herein as a polypeptide having phospholipase activity that is in its final form following translation and any post-translational modifications, such as N-terminal processing, C-terminal truncation, glycosylation, phosphorylation, etc. For a specific gene, the mature polypeptide may vary depending on which host is used to produce the polypeptide.

Mature polypeptide coding sequence: The term “mature polypeptide coding sequence” is defined herein as a nucleotide sequence that encodes a mature polypeptide having phospholipase activity.

Identity: The relatedness between two amino acid sequences or between two nucleotide sequences is described by the parameter “identity”. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:
(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; http://emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:
(Identical Deoxyribonucleotides×100)/(Length of Alignment−Total Number of Gaps in Alignment)

Homologous sequence: The term “homologous sequence” is defined herein as a predicted polypeptide that gives an E value (or expectancy score) of less than 0.001 in a tfasty search (Pearson, W. R., 1999, in Bioinformatics Methods and Protocols, S. Misener and S. A. Krawetz, ed., pp. 185-219) with the Tuber borchii phospholipase A2 (SEQ ID NO:2). Alternatively, the term “homologous sequence” is defined herein as a nucleotide sequence/polypeptide sequence having of identity to the mature polypeptide encoding part of SEQ ID NO: 1 or to the mature polypeptide of SEQ ID NO: 2 or SEQ ID NO: 3, of at least 75%, preferably at least 80%, more preferably at least 85%, more preferably at least 90%, more preferably at least 91%, more preferably at least 92%, even more preferably at least 93%, most preferably at least 94%, and even most preferably at least 95%, such as at least 96%, at least 97%, at least 98%, or even at least 99%.

Polypeptide fragment: The term “polypeptide fragment” is defined herein as a polypeptide having one or more (several) amino acids deleted from the amino and/or carboxyl terminus of the mature polypeptide; or a homologous sequence thereof; wherein the fragment has phospholipase activity. In one aspect, a fragment contains at least 90 amino acid residues, more preferably at least 100 amino acid residues, and most preferably at least 110 amino acid residues of the mature polypeptide or a homologous sequence thereof.

Subsequence: The term “subsequence” is defined herein as a polynucleotide sequence having one or more (several) nucleotides deleted from the 5′ and/or 3′ end of the mature polypeptide coding sequence; or a homologous sequence thereof; wherein the subsequence encodes a polypeptide fragment having phospholipase activity.

Allelic variant: The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Coding sequence: When used herein the term “coding sequence” means a polynucleotide, which directly specifies the amino acid sequence of its polypeptide product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

cDNA: The term “cDNA” is defined herein as a DNA molecule that can be prepared by reverse transcription from a mature, spliced, mRNA molecule obtained from a eukaryotic cell. cDNA lacks intron sequences that are usually present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.

Nucleic acid construct: The term “nucleic acid construct” as used herein refers to a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Control sequences: The term “control sequences” is defined herein to include all components necessary for the expression of a polynucleotide encoding a polypeptide of the present invention. Each control sequence may be native or foreign to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Operably linked: The term “operably linked” denotes herein a configuration in which a control sequence is placed at an appropriate position relative to the coding sequence of the polynucleotide sequence such that the control sequence directs the expression of the coding sequence of a polypeptide.

Expression: The term “expression” includes any step involved in the production of the polypeptide including, but not limited to, transcription, post-transcriptional modification, translation, post-translational modification, and secretion.

Expression vector: The term “expression vector” is defined herein as a linear or circular DNA molecule that comprises a polynucleotide encoding a polypeptide of the present invention and is operably linked to additional nucleotides that provide for its expression.

Host cell: The term “host cell”, as used herein, includes any cell type that is susceptible to transformation, transfection, transduction, and the like with a nucleic acid construct or expression vector comprising a polynucleotide of the present invention.

Improved property: The term “improved property” is defined herein as a characteristic associated with a variant phospholipase that is improved compared to the parent phospholipase. Such improved properties include, but are not limited to, altered temperature-dependent activity profile, thermostability, pH activity, pH stability, substrate specificity, product specificity, and chemical stability. Methods for measuring these properties are well known in the art. Thermostability can be measured, e.g., by Differential Scanning calorimetry (DSC).

Improved product specificity: The term “improved product specificity” is defined herein as a variant phospholipase displaying an altered product profile relative to the parent in which the altered product profile improves the performance of the variant in a given application relative to the parent. The term “product profile” is defined herein as the chemical composition of the reaction products produced by enzymatic hydrolysis. In an embodiment, the improved product specificity is an increased ratio of activity against egg phosphatidyl ethanolamine (PE) to egg phosphatidyl choline (PC) (i.e., PE/PC ratio) when compared to the parent.

Cake: The term “cake” is defined herein as any baked product based on flour and a chemical leavening. The term “cake batter” is defined herein as the sum of cake ingredients before mixing and/or baking. In a cake batter conventional ingredients and their typical amounts (in % by weight of the batter) are:

protein, generally egg yolk lecithin, e.g. in the form of whole eggs, egg yolks, or egg powder: 0.6-3% of egg lecithin or 10-50% of whole eggs.

Flour (untreated, heat treated, chlorinated): 15-30%

Starch (modified, native): 0-10%

Sugar: 15-25%

Emulsifier (mono and diglycerides of fatty acids, propylene glycol esters of fatty acids, lactic acid esters of mono and diglycerides of fatty acids, sodium stearoyl-2-lactylate): 0.1-1

Baking powder (containing soda and acid or acidic salts): 0.5-1%

Hydrocolloids (Locust bean gum, guar gum, tara gum, xanthan gum, carrageenan, acacia gum, cellulose, modified cellulose, pectin): 0-1%

Vegetable fat (ex. oil, margarine, shortening, fat paste, powdered fat): 5-30%

Water: up to 100%

### Improved Cake Properties:

Improved cake properties include improved properties of volume, and texture, e.g., cohesiveness, springiness, and resiliency of the baked product.

Improved volume of the cake may be measured as the volume of cake without a tin divided by the mass of the same cake measured by rape seed displacement method, which is well known in the art. The unit for specific volume is milliliter per gram.

Improved texture of a cake may be measured as described in Bourne M. C. (2002), 2 ed., Food Texture and Viscosity: Concept and Measurement, Academic Press.

Improved cohesiveness and springiness of a cake may be measured as follows: Two consecutive deformations of a cylindrical cake crumb sample (45 mm) performed with a cylindrical probe (100 mm) with a maximum deformation of 50% of the initial height of the product are performed at a deformation speed of 2 mm/second and waiting time between consecutive deformations of 3 seconds. Force is recorded as a function of time. Cohesiveness is calculated as the ratio between the area under the second deformation curve (downwards+upwards) and the area under the first deformation curve (downwards+upwards). Springiness is calculated as the ratio of the height of the decompression of the second deformation to the height of the decompression of the first deformation with 3 seconds waiting time between deformations. Resiliency is calculated as the ratio between the area under the first upward curve and the first downward curve following deformation.

Improved elasticity of a cake may be measured as follows: Penetration of cake crumb with a cylindrical probe (25 mm) until a total deformation of 25% of the initial height of the sample, at a deformation speed of 2 mm/second and keeping the target deformation constant during 20 seconds. Force is registered as a function of time. Elasticity is calculated as the ratio (expressed in percent) between the force measured after 20 seconds at constant deformation to the force applied to obtain the target deformation.

Improved cake properties can be determined by comparing a cake prepared using the phospholipase of the present invention with a control cake prepared under the same conditions (e.g., same recipe), but without the phospholipase treatment.

In another embodiment, the phospholipase variants are used to obtain commercially suitable cake, such as, cakes having a suitable volume, texture, cohesiveness, springiness, and elasticity, when using a reduced amount of egg in the cake recipe. The properties of cake (e.g., volume and texture, including cohesiveness, springiness and elasticity) tend to deteriorate when the amount of egg in the cake recipe is reduced. The variant phospholipases of the present invention may be used to counteract the deterioration by adding the variant phospholipases to the cake batter, preferably, in combination with a non-egg phospholipid or protein source. Accordingly, variant phospholipases may produce commercially acceptable cake properties when the amount of eggs in the cake recipe is reduced, such as, a reduction of amount of eggs (measured by either the egg lecithin or as whole eggs) used in the cake recipe by at least 10%, at least 20%, at least 30%, at least 40%, at least 50%, at least 60%, at least 70%, at least 80%, at least 90%, or at least 95% as compared to a conventional cake recipe. Conventional cake recipes generally use 0.3-1.5% by weight of egg lecithin or 5-25% by weight of whole eggs.

### Conventions for Designation of Variants

For purposes of the present invention, the amino acid sequence of the phospholipase disclosed in the mature polypeptide of SEQ ID NO: 2 is used to determine the corresponding amino acid residue in another phospholipase (variant or parent). For purposes of numbering, the mature polypeptide of SEQ ID NO:2 are amino acids 91 to 210 of the propeptide of SEQ ID NO: 2. SIGNALIP3.0 program that predicts amino acids 1 to 19 of SEQ ID NO: 2 is a signal peptide. For purposes of numbering, the first amino acid of the mature polypeptide is designated by the number or position 1, and accordingly, in accordance with the phospholipase variants of the present invention, the mature polypeptide of SEQ ID NO:2 (and the numbering of amino acids) is:

SPASDTDRLL YSTSMPAFLT AKRNKNPGNL DWSDDGCSNS PDRPAGFNFL

DSCKRHDFGY RNYKKQRRFT EPNRKRIDDN FKKDLYNECA KYSGLQSWKG

VACRKIANTY YDAVRSFGWL

The amino acid sequence of another phospholipase is aligned with the amino acid sequence of the phospholipase disclosed in the mature polypeptide of SEQ ID NO: 2, and based on the alignment the amino acid position number corresponding to any amino acid residue in the amino acid sequence of the phospholipase disclosed in mature polypeptide of SEQ ID NO: 2 can be determined. An alignment of polypeptide sequences may be made, for example, using “ClustalW” (Thompson, J. D., Higgins, D. G. and Gibson, T. J., 1994, CLUSTAL W: Improving the sensitivity of progressive multiple sequence alignment through sequence weighting, positions-specific gap penalties and weight matrix choice, Nucleic Acids Research 22: 4673-4680). An alignment of DNA sequences may be done using the polypeptide alignment as a template, replacing the amino acids with the corresponding codon from the DNA sequence.

Pairwise sequence comparison algorithms in common use are adequate to detect similarities between polypeptide sequences that have not diverged beyond the point of approximately 20-30% sequence identity (Doolittle, 1992, Protein Sci. 1: 191-200; Brenner et al., 1998, Proc. Natl. Acad. Sci. USA 95, 6073-6078). However, truly homologous polypeptides with the same fold and similar biological function have often diverged to the point where traditional sequence-based comparison fails to detect their relationship (Lindahl and Elofsson, 2000, J. Mol. Biol. 295: 613-615). Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide of interest has one or more (several) representatives in the protein structure databases. Programs such as GenTHREADER (Jones 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide of interest, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33:88-96) or combinatorial extension (Shindyalov and Bourne, 1998, Protein Eng. 11:739-747), and implementations of these algorithms can additionally be utilized to query structure databases with a structure of interest in order to discover possible structural homologs (e.g. Holm and Park, 2000, Bioinformatics 16:566-567). These structural alignments can be used to predict the structurally and functionally corresponding amino acid residues in proteins within the same structural superfamily. This information, along with information derived from homology modeling and profile searches, can be used to predict which residues to mutate when moving mutations of interest from one protein to a close or remote homolog.

In describing the various phospholipase variants of the present invention, the nomenclature described below is adapted for ease of reference. In all cases, the accepted IUPAC single letter or triple letter amino acid abbreviation is employed.

Substitutions. For an amino acid substitution, the following nomenclature is used: Original amino acid, position, substituted amino acid or position and substituted amino acid. Accordingly, the substitution of serine with cysteine at position 52 is designated as “Ser52Cys” or “S520”, alternatively, “52Cys” or “52C”. Multiple mutations are separated by addition marks (“+”), e.g., “S520 +D84C” or “52C +84C”, representing mutations at positions 52 and 84 substituting serine (s) with cysteine (C), and aspartic acid (D) with cysteine (C), respectively. Alternative substitution at a position are identified by a comma, e.g., Ser33Glu, Asp or S33E,D represents a substitution of serine (S) with either glutamic acid (E) or aspartic acid (D), alternatively 33E,D.

Deletions. For an amino acid deletion, the following nomenclature is used: Original amino acid, position* or simply position*. Accordingly, the deletion of serine at position 52 is designated as “Ser52*” or “S52*”, alternatively, “52*”. Multiple deletions are separated by addition marks (“+”), e.g., “Ser52* +Asp84*” or “S52* +D84*”.

Insertions. For an amino acid insertion, the following nomenclature is used: Original amino acid, position, original amino acid, new inserted amino acid or position and new inserted amino acid. Accordingly the insertion of lysine after serine at position 52 is designated “Ser52SerLys” or “S52SK”, alternatively, “52SerLys” or “52SK”. Multiple insertions of amino acids are designated [Original amino acid, position, original amino acid, new inserted amino acid #1, new inserted amino acid #2; etc. or position and new inserted amino acid #1, new inserted amino acid #2; etc.]. For example, the insertion of lysine and alanine after serine at position 52 is indicated as “Ser52SerLysAla” or “S52SKA” or “52SLK”.

In such cases the inserted amino acid residue(s) are numbered by the addition of lower case letters to the position number of the amino acid residue preceding the inserted amino acid residue(s). In the above example the sequences would thus be:

| Parent: | Variant: |
|---|---|
| 52 | 52 52a 52b |
| S | S-K-A |

N and/or C Terminal Extensions. For an amino acid insertion, the following nomenclature is used: Original N and/or C terminal amino acid, position, plus amino acid extension(s) or original N and/or C terminal position plus amino acid extension(s). For example, the extension of the C-terminus of the phospholipase can be designated by the following L12OLDATPG indicating an addition of amino acids DATPG to the C-terminus. Alternatively, an extension may be indicated by adding additional amino acid position numbering. For example, the same extension of the C-terminus of the phospholipase can be designated by the following L120+121D+122A+123T+124P+125G, and when combined with an alteration at the terminal amino acid can be designated by the following L120D+121D+122A+123T+124P+125G.

### Parent Phospholipase:

The parent phospholipase includes a polypeptide comprising or consisting of an amino acid sequence having the amino acid sequence of the mature polypeptide of SEQ ID NO:2 or a polypeptide comprising an amino acid sequence having at least 50% identity with the mature polypeptide of SEQ ID NO:2; such as, at least 60% identity with the mature polypeptide of SEQ ID NO: 2, at least 65% identity with the mature polypeptide of SEQ ID NO:2, at least 70% identity with the mature polypeptide of SEQ ID NO:2, at least 75% identity with the mature polypeptide of SEQ ID NO:2, at least 80% identity with the mature polypeptide of SEQ ID NO:2, at least 85% identity with the mature polypeptide of SEQ ID NO: 2, at least 90% identity with the mature polypeptide of SEQ ID NO:2, at least 91% identity with the mature polypeptide of SEQ ID NO:2, at least 92% identity with the mature polypeptide of SEQ ID NO:2, at least 93% identity with the mature polypeptide of SEQ ID NO:2, at least 94% identity with the mature polypeptide of SEQ ID NO:2, at least 95% identity with the mature polypeptide of SEQ ID NO:2, at least 96% identity with the mature polypeptide of SEQ ID NO:2, at least 97% identity with the mature polypeptide of SEQ ID NO:2, at least 98% identity with the mature polypeptide of SEQ ID NO:2, or at least 99% identity with the mature polypeptide of SEQ ID NO:2.

The parent phospholipase includes a polypeptide comprising or consisting of an amino acid sequence having the amino acid sequence of the mature polypeptide of SEQ ID NO:3 or a polypeptide comprising an amino acid sequence having at least 50% identity with the mature polypeptide of SEQ ID NO:3; such as, at least 60% identity with the mature polypeptide of SEQ ID NO: 3, at least 65% identity with the mature polypeptide of SEQ ID NO:3, at least 70% identity with the mature polypeptide of SEQ ID NO:3, at least 75% identity with the mature polypeptide of SEQ ID NO:3, at least 80% identity with the mature polypeptide of SEQ ID NO:3, at least 85% identity with the mature polypeptide of SEQ ID NO: 3, at least 90% identity with the mature polypeptide of SEQ ID NO: 3, at least 91% identity with the mature polypeptide of SEQ ID NO:3, at least 92% identity with the mature polypeptide of SEQ ID NO:3, at least 93% identity with the mature polypeptide of SEQ ID NO:3, at least 94% identity with the mature polypeptide of SEQ ID NO:3, at least 95% identity with the mature polypeptide of SEQ ID NO:3, at least 96% identity with the mature polypeptide of SEQ ID NO:3, at least 97% identity with the mature polypeptide of SEQ ID NO:3, at least 98% identity with the mature polypeptide of SEQ ID NO:3, or at least 99% identity with the mature polypeptide of SEQ ID NO:3.

In one aspect, the parent phospholipase is a polypeptide having an amino acid sequence that differs from the mature polypeptide of SEQ ID NO:2 or the mature polypeptide of SEQ ID NO:2 by thirty amino acids, twenty-nine amino acids, twenty-eight amino acids, twenty-seven amino acids, twenty-six amino acids, twenty-five amino acids, twenty-four amino acids, twenty-three amino acids, twenty-two amino acids, twenty-one amino acids, twenty amino acids, nineteen amino acids, eighteen amino acids, seventeen amino acids, sixteen amino acids, fifteen amino acids, fourteen amino acids, thirteen amino acids, twelve amino acids, eleven amino acids, ten amino acids, nine amino acids, eight amino acids, seven amino acids, six amino acids, five amino acids, four amino acids, three amino acids, two amino acids, or one amino acid. Examples of amino acid differences includes changes which are a minor nature, such as, conservative amino acid substitutions and other substitutions that do not significantly affect the three-dimensional folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, or protein A (Nilsson et al., 1985, EMBO J. 4: 1075; Nilsson et al., 1991, Methods Enzymol. 198: 3. See, also, in general, Ford et al., 1991, Protein Expression and Purification 2: 95-107. Although the changes described above preferably are of a minor nature, such changes may also be of a substantive nature such as fusion of larger polypeptides of up to 300 amino acids or more both as amino- or carboxyl-terminal extensions.

The parent phospholipase preferably comprises or consists of the mature polypeptide of the amino acid sequence of SEQ ID NO: 2, the mature polypeptide of SEQ ID NO:3, or an allelic variant thereof; or a fragment thereof having phospholipase activity. A fragment contains at least 90 amino acid residues, more preferably at least 100 amino acid residues, and most preferably at least 110 amino acid residues of the mature polypeptide of SEQ ID NO:2, the mature polypeptide of SEQ ID NO:3 or homologous sequences thereof.

The parent phospholipase may be obtained from microorganisms of any genus. For purposes of the present invention, the term “obtained from” as used herein in connection with a given source shall mean that the parent phospholipase encoded by a polynucleotide is produced by the source or by a cell in which the polynucleotide from the source has been inserted. In one aspect, the parent phospholipase is secreted extracellularly.

The parent phospholipase may be a fungal phospholipase. In another aspect, the parent phospholipase is obtained from the genus Tuber, such as, the species Tuber borchii or Tuber albidum. Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). In another embodiment, the parent phospholipase is the Tuber borchii phospholipase A2 or Tuber albidum phospholipase A2, such as described in Soragni et al., 2001, EMBO J. 20: 5079-5090 and US Patent Publication 20070092945, which are hereby incorporated by reference. It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

In another embodiment, the parent phospholipase is the Tuber borchii phospholipase A2 comprising an amino acid sequence of the mature polypeptide of SEQ ID NO:3 or the Tuber albidum phospholipase A2 comprising an amino acid sequence of the mature polypeptide of SEQ ID NO:2.

The parent phospholipase may also be identified and obtained from other sources including microorganisms isolated from nature (e.g., soil, composts, water, etc.) or DNA samples obtained directly from natural materials (e.g., soil, composts, water, etc,) using the above-mentioned probes. Techniques for isolating microorganisms and DNA directly from natural habitats are well known in the art. The polynucleotide encoding a phospholipase may then be derived by similarly screening a genomic or cDNA library of another microorganism or mixed DNA sample. Once a polynucleotide encoding a phospholipase has been detected with suitable probe(s) as described herein, the sequence may be isolated or cloned by utilizing techniques that are known to those of ordinary skill in the art (see, e.g., J. Sambrook, E. F. Fritsch, and T. Maniatus, 1989, Molecular Cloning, A Laboratory Manual, 2d edition, Cold Spring Harbor, N.Y.).

The parent phospholipase can also include fused polypeptides or cleavable fusion polypeptides in which another polypeptide is fused at the N-terminus or the C-terminus of the polypeptide or fragment thereof. A fused polypeptide is produced by fusing a polynucleotide (or a portion thereof) encoding another polypeptide to a polynucleotide (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

### Variant Phospholipases:

In the present invention, the isolated variants of a parent phospholipase comprise or consists of an alteration at one or more positions corresponding to positions 1, 6, 30, 31, 33, 38, 39, 42, 43, 44, 45, 47, 52, 59, 61, 64, 65, 77, 84, 102, 106, 110, 116, 119, or 120 of the mature polypeptide of SEQ ID NO: 2, wherein the variants has phospholipase activity. In an embodiment, the variant comprises an amino acid sequence having at least 50% identity with of the mature polypeptide SEQ ID NO:2; such as, at least 60% identity with of the mature polypeptide SEQ ID NO: 2, at least 65% identity with of the mature polypeptide SEQ ID NO:2, at least 70% identity with of the mature polypeptide SEQ ID NO:2, at least 75% identity with of the mature polypeptide SEQ ID NO:2, at least 80% identity with of the mature polypeptide SEQ ID NO:2, at least 85% identity with of the mature polypeptide SEQ ID NO: 2, at least 90% identity with of the mature polypeptide SEQ ID NO:2, at least 91% identity with of the mature polypeptide SEQ ID NO:2, at least 92% identity with of the mature polypeptide SEQ ID NO:2, at least 93% identity with of the mature polypeptide SEQ ID NO:2, at least 94% identity with of the mature polypeptide SEQ ID NO:2, at least 95% identity with of the mature polypeptide SEQ ID NO:2, at least 96% identity with of the mature polypeptide SEQ ID NO:2, at least 97% identity with of the mature polypeptide SEQ ID NO:2, at least 98% identity with of the mature polypeptide SEQ ID NO:2, or at least 99% identity with of the mature polypeptide SEQ ID NO:2.

In an embodiment, the variant or consists of an alteration at one or more positions corresponding to positions 1, 6, 30, 31, 33, 38, 39, 42, 43, 44, 45, 47, 52, 59, 61, 64, 65, 77, 84, 102, 106, 110, 116, 119, or 120 of the mature polypeptide of SEQ ID NO: 2, has phospholipase activity, and comprises an amino acid sequence having at least 50% identity with of the mature polypeptide SEQ ID NO:3; such as, at least 60% identity with of the mature polypeptide SEQ ID

NO:3, at least 65% identity with of the mature polypeptide SEQ ID NO:3, at least 70% identity with of the mature polypeptide SEQ ID NO:3, at least 75% identity with of the mature polypeptide SEQ ID NO:3, at least 80% identity with of the mature polypeptide SEQ ID NO:3, at least 85% identity with of the mature polypeptide SEQ ID NO:3, at least 90% identity with of the mature polypeptide SEQ ID NO:3, at least 91% identity with of the mature polypeptide SEQ ID NO:3, at least 92% identity with of the mature polypeptide SEQ ID NO:3, at least 93% identity with of the mature polypeptide SEQ ID NO:3, at least 94% identity with of the mature polypeptide SEQ ID NO:3, at least 95% identity with of the mature polypeptide SEQ ID NO:3, at least 96% identity with of the mature polypeptide SEQ ID NO:3, at least 97% identity with of the mature polypeptide SEQ ID NO:3, at least 98% identity with of the mature polypeptide SEQ ID NO:3, or at least 99% identity with of the mature polypeptide SEQ ID NO:3.

The present invention is also directed to the use of phospholipase variants which comprise or consists of an alteration at one or more positions corresponding to positions 1, 6, 30, 31, 33, 38, 39, 42, 43, 44, 45, 47, 52, 59, 61, 64, 65, 77, 84, 102, 106, 110, 116, 119, or 120 of SEQ ID NO:2, wherein the variants has phospholipase activity, and comprises an amino acid sequence having at least 50% identity with the Tuber borchii phospholipase A2 or Tuber albidum phospholipase A2; such as, at least 60% identity with the Tuber borchii phospholipase A2 or Tuber albidum phospholipase A2, at least 65% identity with the Tuber borchii phospholipase A2 or Tuber albidum phospholipase A2, at least 70% identity with the Tuber borchii phospholipase A2 or Tuber albidum phospholipase A2, at least 75% identity with the Tuber borchii phospholipase A2 or Tuber albidum phospholipase A2, at least 80% identity with the Tuber borchii phospholipase A2 or Tuber albidum phospholipase A2, at least 85% identity with the Tuber borchii phospholipase A2 or Tuber albidum phospholipase A2, at least 90% identity with the Tuber borchii phospholipase A2 or Tuber albidum phospholipase A2, at least 91% identity with the Tuber borchii phospholipase A2 or Tuber albidum phospholipase A2, at least 92% identity with the Tuber borchii phospholipase A2 or Tuber albidum phospholipase A2, at least 93% identity with the Tuber borchii phospholipase A2 or Tuber albidum phospholipase A2, at least 94% identity with the Tuber borchii phospholipase A2 or Tuber albidum phospholipase A2, at least 95% identity with the Tuber borchii phospholipase A2 or Tuber albidum phospholipase A2, at least 96% identity with the Tuber borchii phospholipase A2 or Tuber albidum phospholipase A2, at least 97% identity with the Tuber borchii phospholipase A2 or Tuber albidum phospholipase A2, at least 98% identity with the Tuber borchii phospholipase A2 or Tuber albidum phospholipase A2, or at least 99% identity with the Tuber borchii phospholipase A2 or Tuber albidum phospholipase A2.

In one aspect, the phospholipase variant is a polypeptide having phospholipase activity, and having an amino acid sequence that differs from the mature polypeptide of SEQ ID NO:2 or the mature polypeptide of SEQ ID NO:3 by thirty amino acids, twenty-nine amino acids, twenty-eight amino acids, twenty-seven amino acids, twenty-six amino acids, twenty-five amino acids, twenty-four amino acids, twenty-three amino acids, twenty-two amino acids, twenty-one amino acids, twenty amino acids, nineteen amino acids, eighteen amino acids, seventeen amino acids, sixteen amino acids, fifteen amino acids, fourteen amino acids, thirteen amino acids, twelve amino acids, eleven amino acids, ten amino acids, nine amino acids, eight amino acids, seven amino acids, six amino acids, five amino acids, four amino acids, three amino acids, two amino acids, or one amino acid.

In one aspect, the variant phospholipase comprises an alteration (substitution, deletion or insertion) at a position corresponding to position 1 (using the mature polypeptide of SEQ ID NO:2 for numbering). In one embodiment, the variant phospholipase comprises a substitution of an amino acid at a position corresponding to position 1 (using the mature polypeptide of SEQ ID NO:2 for numbering) with Ala, Arg, Asn, Asp, Cys, Gln, Glu, Gly, His, Ile, Leu, Lys, Met, Phe, Pro, Thr, Trp, Tyr, or Val.

In one aspect, the variant phospholipase comprises an alteration (substitution, deletion or insertion) at a position corresponding to position 6 (using the mature polypeptide of SEQ ID NO:2 for numbering). In one embodiment, the variant phospholipase comprises a substitution of an amino acid at a position corresponding to position 6 (using the mature polypeptide of SEQ ID NO:2 for numbering) with Ala, Arg, Asn, Asp, Cys, Gln, Glu, Gly, His, Ile, Leu, Lys, Met, Phe, Pro, Ser, Trp, Tyr, or Val.

In one aspect, the variant phospholipase comprises an alteration (substitution, deletion or insertion) at a position corresponding to position 30 (using the mature polypeptide of SEQ ID NO:2 for numbering). In one embodiment, the variant phospholipase comprises a substitution of an amino acid at a position corresponding to position 30 (using the mature polypeptide of SEQ ID NO:2 for numbering) with Ala, Arg, Asn, Asp, Cys, Gln, Glu, Gly, His, Ile, Lys, Met, Phe, Pro, Ser, Thr, Trp, Tyr, or Val.

In one aspect, the variant phospholipase comprises an alteration (substitution, deletion or insertion) at a position corresponding to position 31 (using the mature polypeptide of SEQ ID NO:2 for numbering). In one embodiment, the variant phospholipase comprises a substitution of an amino acid at a position corresponding to position 31 (using the mature polypeptide of SEQ ID NO:2 for numbering) with Ala, Arg, Asn, Cys, Gln, Glu, Gly, His, Ile, Leu, Lys, Met, Phe, Pro, Ser, Thr, Trp, Tyr, or Val.

In one aspect, the variant phospholipase comprises an alteration (substitution, deletion or insertion) at a position corresponding to position 33 (using the mature polypeptide of SEQ ID NO:2 for numbering). In one embodiment, the variant phospholipase comprises a substitution of an amino acid at a position corresponding to position 33 (using the mature polypeptide of SEQ ID NO:2 for numbering) with Ala, Arg, Asn, Asp, Cys, Gln, Glu, Gly, His, Ile, Leu, Lys, Met, Phe, Pro, Thr, Trp, Tyr, or Val.

In one aspect, the variant phospholipase comprises an alteration (substitution, deletion or insertion) at a position corresponding to position 38 (using the mature polypeptide of SEQ ID NO:2 for numbering). In one embodiment, the variant phospholipase comprises a substitution of an amino acid at a position corresponding to position 38 (using the mature polypeptide of SEQ ID NO:2 for numbering) with Ala, Arg, Asn, Asp, Cys, Gln, Glu, Gly, His, Ile, Leu, Lys, Met, Phe, Pro, Thr, Trp, Tyr, or Val.

In one aspect, the variant phospholipase comprises an alteration (substitution, deletion or insertion) at a position corresponding to position 39 (using the mature polypeptide of SEQ ID NO:2 for numbering). In one embodiment, the variant phospholipase comprises a substitution of an amino acid at a position corresponding to position 39 (using the mature polypeptide of SEQ ID NO:2 for numbering) with Ala, Arg, Asp, Cys, Gln, Glu, Gly, His, Ile, Leu, Lys, Met, Phe, Pro, Ser, Thr, Trp, Tyr, or Val.

In one aspect, the variant phospholipase comprises an alteration (substitution, deletion or insertion) at a position corresponding to position 42 (using the mature polypeptide of SEQ ID NO:2 for numbering). In one embodiment, the variant phospholipase comprises a substitution of an amino acid at a position corresponding to position 42 (using the mature polypeptide of SEQ ID NO:2 for numbering) with Ala, Arg, Asn, Cys, Gln, Glu, Gly, His, Ile, Leu, Lys, Met, Phe, Pro, Ser, Thr, Trp, Tyr, or Val.

In one aspect, the variant phospholipase comprises an alteration (substitution, deletion or insertion) at a position corresponding to position 43 (using the mature polypeptide of SEQ ID NO:2 for numbering). In one embodiment, the variant phospholipase comprises a substitution of an amino acid at a position corresponding to position 43 (using the mature polypeptide of SEQ ID NO:2 for numbering) with Ala, Asn, Asp, Cys, Gln, Glu, Gly, His, Ile, Leu, Lys, Met, Phe, Pro, Ser, Thr, Trp, Tyr, or Val.

In one aspect, the variant phospholipase comprises an alteration (substitution, deletion or insertion) at a position corresponding to position 44 (using the mature polypeptide of SEQ ID NO:2 for numbering). In one embodiment, the variant phospholipase comprises a substitution of an amino acid at a position corresponding to position 44 (using the mature polypeptide of SEQ ID NO:2 for numbering) with Ala, Arg, Asn, Asp, Cys, Gln, Glu, Gly, His, Ile, Leu, Lys, Met, Phe, Ser, Thr, Trp, Tyr, or Val.

In one aspect, the variant phospholipase comprises an alteration (substitution, deletion or insertion) at a position corresponding to position 45 (using the mature polypeptide of SEQ ID NO:2 for numbering). In one embodiment, the variant phospholipase comprises a substitution of an amino acid at a position corresponding to position 45 (using the mature polypeptide of SEQ ID NO:2 for numbering) with Arg, Asn, Asp, Cys, Gln, Glu, Gly, His, Ile, Leu, Lys, Met, Phe, Pro, Ser, Thr, Trp, Tyr, or Val.

In one aspect, the variant phospholipase comprises an alteration (substitution, deletion or insertion) at a position corresponding to position 47 (using the mature polypeptide of SEQ ID NO:2 for numbering). In one embodiment, the variant phospholipase comprises a substitution of an amino acid at a position corresponding to position 47 (using the mature polypeptide of SEQ ID NO:2 for numbering) with Ala, Arg, Asn, Asp, Cys, Gln, Glu, Gly, His, Ile, Leu, Lys, Met, Pro, Ser, Thr, Trp, Tyr, or Val.

In one aspect, the variant phospholipase comprises an alteration (substitution, deletion or insertion) at a position corresponding to position 52 (using the mature polypeptide of SEQ ID NO:2 for numbering). In one embodiment, the variant phospholipase comprises a substitution of an amino acid at a position corresponding to position 52 (using the mature polypeptide of SEQ ID NO:2 for numbering) with Ala, Arg, Asn, Asp, Cys, Gln, Glu, Gly, His, Ile, Leu, Lys, Met, Phe, Pro, Thr, Trp, Tyr, or Val.

In one aspect, the variant phospholipase comprises an alteration (substitution, deletion or insertion) at a position corresponding to position 59 (using the mature polypeptide of SEQ ID NO:2 for numbering). In one embodiment, the variant phospholipase comprises a substitution of an amino acid at a position corresponding to position 59 (using the mature polypeptide of SEQ ID NO:2 for numbering) with Ala, Arg, Asn, Asp, Cys, Gln, Glu, His, Ile, Leu, Lys, Met, Phe, Pro, Ser, Thr, Trp, Tyr, or Val.

In one aspect, the variant phospholipase comprises an alteration (substitution, deletion or insertion) at a position corresponding to position 61 (using the mature polypeptide of SEQ ID NO:2 for numbering). In one embodiment, the variant phospholipase comprises a substitution of an amino acid at a position corresponding to position 61 (using the mature polypeptide of SEQ ID NO:2 for numbering) with Ala, Asn, Asp, Cys, Gln, Glu, Gly, His, Ile, Leu, Lys, Met, Phe, Pro, Ser, Thr, Trp, Tyr, or Val.

In one aspect, the variant phospholipase comprises an alteration (substitution, deletion or insertion) at a position corresponding to position 64 (using the mature polypeptide of SEQ ID NO:2 for numbering). In one embodiment, the variant phospholipase comprises a substitution of an amino acid at a position corresponding to position 64 (using the mature polypeptide of SEQ ID NO:2 for numbering) with Ala, Arg, Asn, Asp, Cys, Gln, Glu, Gly, His, Ile, Leu, Met, Phe, Pro, Ser, Thr, Trp, Tyr, or Val.

In one aspect, the variant phospholipase comprises an alteration (substitution, deletion or insertion) at a position corresponding to position 65 (using the mature polypeptide of SEQ ID NO:2 for numbering). In one embodiment, the variant phospholipase comprises a substitution of an amino acid at a position corresponding to position 65 (using the mature polypeptide of SEQ ID NO:2 for numbering) with Ala, Arg, Asn, Asp, Cys, Gln, Glu, Gly, His, Ile, Leu, Met, Phe, Pro, Ser, Thr, Trp, Tyr, or Val.

In one aspect, the variant phospholipase comprises an alteration (substitution, deletion or insertion) at a position corresponding to position 77 (using the mature polypeptide of SEQ ID NO:2 for numbering). In one embodiment, the variant phospholipase comprises a substitution of an amino acid at a position corresponding to position 77 (using the mature polypeptide of SEQ ID NO:2 for numbering) with Ala, Arg, Asn, Asp, Cys, Gln, Glu, Gly, His, Leu, Lys, Met, Phe, Pro, Ser, Thr, Trp, Tyr, or Val.

In one aspect, the variant phospholipase comprises an alteration (substitution, deletion or insertion) at a position corresponding to position 84 (using the mature polypeptide of SEQ ID NO:2 for numbering). In one embodiment, the variant phospholipase comprises a substitution of an amino acid at a position corresponding to position 84 (using the mature polypeptide of SEQ ID NO:2 for numbering) with Ala, Arg, Asn, Cys, Gln, Glu, Gly, His, Ile, Leu, Lys, Met, Phe, Pro, Ser, Thr, Trp, Tyr, or Val.

In one aspect, the variant phospholipase comprises an alteration (substitution, deletion or insertion) at a position corresponding to position 102 (using the mature polypeptide of SEQ ID NO:2 for numbering). In one embodiment, the variant phospholipase comprises a substitution of an amino acid at a position corresponding to position 102 (using the mature polypeptide of SEQ ID

NO:2 for numbering) with Arg, Asn, Asp, Cys, Gln, Glu, Gly, His, Ile, Leu, Lys, Met, Phe, Pro, Ser, Thr, Trp, Tyr, or Val.

In one aspect, the variant phospholipase comprises an alteration (substitution, deletion or insertion) at a position corresponding to position 106 (using the mature polypeptide of SEQ ID NO:2 for numbering). In one embodiment, the variant phospholipase comprises a substitution of an amino acid at a position corresponding to position 106 (using the mature polypeptide of SEQ ID NO:2 for numbering) with Ala, Arg, Asn, Asp, Cys, Gln, Glu, Gly, His, Leu, Lys, Met, Phe, Pro, Ser, Thr, Trp, Tyr, or Val.

In one aspect, the variant phospholipase comprises an alteration (substitution, deletion or insertion) at a position corresponding to position 110 (using the mature polypeptide of SEQ ID NO:2 for numbering). In one embodiment, the variant phospholipase comprises a substitution of an amino acid at a position corresponding to position 110 (using the mature polypeptide of SEQ ID NO:2 for numbering) with Ala, Arg, Asn, Asp, Cys, Gln, Glu, Gly, His, Ile, Leu, Lys, Met, Phe, Pro, Ser, Thr, Trp, or Val.

In one aspect, the variant phospholipase comprises an alteration (substitution, deletion or insertion) at a position corresponding to position 116 (using the mature polypeptide of SEQ ID NO:2 for numbering). In one embodiment, the variant phospholipase comprises a substitution of an amino acid at a position corresponding to position 116 (using the mature polypeptide of SEQ ID NO:2 for numbering) with Ala, Arg, Asn, Asp, Cys, Gln, Glu, Gly, His, Ile, Leu, Lys, Met, Phe, Pro, Thr, Trp, Tyr, or Val.

In one aspect, the variant phospholipase comprises an alteration (substitution, deletion or insertion) at a position corresponding to position 119 (using the mature polypeptide of SEQ ID NO:2 for numbering). In one embodiment, the variant phospholipase comprises a substitution of an amino acid at a position corresponding to position 119 (using the mature polypeptide of SEQ ID NO:2 for numbering) with Ala, Arg, Asn, Asp, Cys, Gln, Glu, Gly, His, Ile, Leu, Lys, Met, Phe, Pro, Ser, Thr, Tyr, or Val.

In one aspect, the variant phospholipase comprises an alteration (substitution, deletion or insertion) at a position corresponding to position 120 (using the mature polypeptide of SEQ ID NO:2 for numbering). In one embodiment, the variant phospholipase comprises a substitution of an amino acid at a position corresponding to position 120 (using the mature polypeptide of SEQ ID NO:2 for numbering) with Ala, Arg, Asn, Asp, Cys, Gln, Glu, Gly, His, Ile, Lys, Met, Phe, Pro, Ser, Thr, Trp, Tyr, or Val.

In another aspect, the variant phospholipase comprises a peptide extension of one or more amino acids at the -N and/or -C terminus of the phospholipase. In one embodiment, the variant comprises or consists of an insertion at the -N and/or -C terminus of the phospholipase of from 1 to 10 amino acids, or 1 to 9 amino acids, or 1 to 8 amino acids, or 1 to 7 amino acids, or 1 to 6 amino acids, 1 to 5 amino acids, or 1 to 4 amino acids, or 1 to 3 amino acids or 2 amino acids or 1 amino acid. In one embodiment, the variant consists of an insertion at the -N and/or -C terminus of the phospholipase of less than 20 amino acids, less than 19 amino acids, less than 18 amino acids, less than 17 amino acids, less than 16 amino acids, less than 15 amino acids, less than 14 amino acids, less than 13 amino acids, less than 12 amino acids, less than 11 amino acids, less than 10 amino acids, less than 9 amino acids, less than 8 amino acids, less than 7 amino acids, less than 6 amino acids, less than 5 amino acids, less than 4 amino acids, less than 3 amino acids, less than 2 amino acids, or 1 amino acid.

The variant phospholipase may also comprise a truncation of one or more amino acid residues at the N and/or C terminus, such as from 1 to 30 amino acid residue deletion, 1 to 20 amino acid residue deletion, 1 to 10 amino acids residue deletion, 1 to 9 amino acid residue deletion of 1 to 8 amino acid residue deletion, 1 to 7 amino acid residue deletion, 1 to 6 amino acid residue deletion, 1 to 5 amino acid residue deletion, 1 to 4 amino acid residue deletion, 1 to 3 amino acid residue deletion 2 amino acid residue deletion or 1 amino acid residue deletion. The alterations described herein may be used in combination, e.g., a substitution at the N and/or C terminal amino acid combined with an N and/or C-terminal peptide extension.

In exemplary embodiments of the phospholipase variants of invention, the variant comprises (using the mature polypeptide of SEQ ID NO:2 for numbering) and one or more (several) alterations selected from the group consisting of

substitution of E or D at position 33 (such as, S33E,D);

substitution of E at position 31 (such as, D31 E);

substitution of E at position 65 (such as, K65E);

substitution of T or D at position 38 (such as S38T,D);

substitution of K at position 39 (such as, N39K);

substitution of Y at position 110 (such as, Y110F);

substitution of L,V or A at position 106 (such as, 1106L,V,A);

substitution of D, F or C at position 45 (such as, A45D,F,C);

substitution of Y at position 47 (such as, F47F);

substitution of E at position 102 (such as, A102E).

substitution of R at position 64 (such as, K64R);

substitution of T at position 116 (such as, S116T)

substitution of G at position 119 (such as W119G); and

substitution of D at position 120 (such L120D);

insertion of at the C-terminus (such as, insertion of D-A-T-P-G at the C-terminus).

In exemplary embodiments of combinations of alterations, the variant comprises (using the mature polypeptide of SEQ ID NO:2 for numbering) one of the following:

a substitution of Y at position 47 plus a substitution of E at position 102 (such as F47Y+A102E); or

a substitution of R at position 64 plus a substitution of G at position 119 plus a substitution of D at position 120 plus an C-terminal extension of DATPG (such as K64R+W119G+L120D+121D+122A+123T+124P+125G).

In another exemplary embodiment, the variant comprises the creation of extra disulfide bridge (using the mature polypeptide of SEQ ID NO:2 for numbering) by making the following alterations:

substitutions of cysteine residues at positions 52 and 84 (such as, S52C+D84C)

substitutions of cysteine residues at positions 59 and 77 (such as, G59C+177C)

substitutions of cysteine residues at positions 1 and 30 (such as, S1C+L30C)

substitution of cysteine residues at positions 6 and 30 (such as, T6C+L30C).

In additional exemplary embodiments of the invention, the variant comprises (using the mature polypeptide of SEQ ID NO:2 for numbering) one or more (several) alterations selected from the group consisting of:

31E (such as D31E);

33C,W,D,M,E,G,A,Y,R,L,Q (such as, S33C,W,D,M,E,G,A,Y,R,L,Q);

38D,A,T (such as S38D,A,T);

39K,C,I,F,L,M,S,P,T,W,R,Q (such as N39K,C,I,F,L,M,S,P,T,W,R,Q);

42V (such as, D42V)

43W (such as, R43W)

44L (such a, P44L)

45D,F,V,L,K,T,G,R,E,C (such as, A45D,F,V,L,K,T,G,R,E,C);

47Y,L,W,R,V,G,C (such as, F47Y,L,W,R,V,G,C);

61C,F,Y,A,V,K,L,N,E,I,S (such as R61C,F,Y,A,V,K,L,N,E,I,S)

64R (such as K65R);

65E (such as, K65E);

77C (such as, 177C);

84C (such as, D84C);

102E,G,H,S (such as, A102E,G,H,S);

106A,V,P,L (such as, 1106A,V,P,L);

110F (such as, Y110F)

116Q,H,R,T,A,L,I,Y,P,F (such as, SI 16Q,H,R,T,A,L,I,Y,P,F)

119V,H,A,R,T,K,L,I,N,G,E,Q,P,C,S,F (such as, W119V,H,A,R,T, K, L, I, N,G,E,Q,P,C,S;F);

120E,S,A,K,H,Y,P,T,V,Q,R,I (such as L120E,S,A,K,H,Y,P,T,V,Q,R,I)

In additional exemplary embodiments of the invention, the variant comprises (using the mature polypeptide of SEQ ID NO:2 for numbering) one of the following alterations:

47Y+102E (such as, F47Y+A102E);

64R+116C (such as, K64R+S116C);

119G+120DDATPG (such as, W119G+L120DDATPG);

119H+120IATRA (such as, W119H+L120IATRA);

119F+120ICNSSL (such as, W119F+L120ICNSSL);

119H+120CNSSLR (such as, W119H+L120CNSSLR);

119H+120IVTRA (such as, W119H+L120IVTRA);

119P+120LCNSSL (such as, W119P+L120LCNSSL);

64R+119G+120DDATPG (such as, K64R+W119G+L120DDATPG);

42V+43W (such as, D42V+R43W);

44L+47L (such as P44L, F47L);

33D+119G (such as, S33D+W119G);

33D+39K+119G (such as, S33D+N39K+W119G);

33D+39K+119N (such as, S33D+N39K+W119N);

31Y+33D+39K+119N (such as, D31Y+S33D+, N39K+W119N)

39K+119G (such as, N39K+W119G)

### Preparation of Variants

Variants of a parent phospholipase can be prepared according to any mutagenesis procedure known in the art, such as site-directed mutagenesis, synthetic gene construction, semi-synthetic gene construction, random mutagenesis, shuffling, etc. Nucliec acids encoding parent phospholipases that may be used to prepare the variants of the present invention include, e.g., the nucleic acid sequence shown as SEQ ID NO:1.

Other nucleic acids encoding parent phospholipases include nucleic acid sequences that hybridize under very low stringency conditions, low stringency conditions, medium stringency conditions, medium-high stringency conditions, high stringency conditions, and very high stringency condition with nucleic acid sequence encoding the mature polypeptide of SEQ ID NO:2 or SEQ ID NO:3, a subsequence thereof or a complementary strand thereof (J. Sambrook, E. F. Fritsch, and T. Maniatus, 1989, Molecular Cloning, A Laboratory Manual, 2d edition, Cold Spring Harbor, N.Y.). A subsequence contains at least 100 contiguous nucleotides or preferably at least 200 contiguous nucleotides. Stringency conditions are defined as prehybridization and hybridization at 42° C. in 5×SSPE, 0.3% SDS, 200 ug/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. For long probes of at least 100 nucleotides in length, the carrier material is finally washed three times each for 15 minutes using 2×SSC, 0.2% SOS preferably at least at 45° C. (very low stringency), more preferably at least at 50° C. (low stringency), more preferably at least at 55° C. (medium stringency), more preferably at least at 60° C. (medium-high stringency), even more preferably at least at 65° (high stringency), and most preferably at least at 70° C. (very high stringency).

Site-directed mutagenesis is a technique in which one or several mutations are created at a defined site in a polynucleotide molecule encoding the parent phospholipase. The technique can be performed in vitro or in vivo.

Synthetic gene construction entails in vitro synthesis of a designed polynucleotide molecule to encode a polypeptide molecule of interest. Gene synthesis can be performed utilizing a number of techniques, such as the multiplex microchip-based technology described by Tian, et. al., (Tian, et. al., Nature 432:1050-1054) and similar technologies wherein olgionucleotides are synthesized and assembled upon photo-programmable microfluidic chips.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent phospholipase and subsequent ligation of an oligonucleotide containing the mutation in the polynucleotide. Usually the restriction enzyme that digests at the plasmid and the oligonucleotide is the same, permitting sticky ends of the plasmid and insert to ligate to one another. See, for example, Scherer and Davis, 1979, Proc. Natl. Acad. Sci. USA 76: 4949-4955; and Barton et al., 1990, Nucleic Acids Research 18: 7349-4966.

Site-directed mutagenesis can be accomplished in vivo by methods known in the art. See, for example, U.S. Patent Application Publication 2004/0171154; Storici et al., 2001, Nature Biotechnology 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants of a parent phospholipase.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204) and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide fragments may then be shuffled.

### Nucleic Acid Constructs

An isolated polynucleotide encoding a phospholipase variant of the present invention may be manipulated in a variety of ways to provide for expression of the variant. Manipulation of the polynucleotide prior to its insertion into a vector may be desirable or necessary depending on the expression vector. The techniques for modifying polynucleotides utilizing recombinant DNA methods are well known in the art.

The control sequence may be an appropriate promoter sequence, which is recognized by a host cell for expression of the polynucleotide. The promoter sequence contains transcriptional control sequences that mediate the expression of the variant phospholipase. The promoter may be any nucleic acid sequence that shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Daria (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the variant phospholipase. Any terminator that is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA that is important for translation by the host cell. The leader sequence is operably linked to the 5′-terminus of the polynucleotide encoding the variant phospholipase. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polypeptide-encoding sequence and, when transcribed, is recognized by the host cell as a signal to add polyadenosine residues to transcribed mRNA. Any polyadenylation sequence that is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

The control sequence may also be a signal peptide coding region that codes for an amino acid sequence linked to the amino terminus of a variant phospholipase and directs the encoded polypeptide into the cell's secretory pathway. The 5′-end of the coding sequence of the polynucleotide may inherently contain a signal peptide coding region naturally linked in translation reading frame with the segment of the coding region that encodes the secreted variant phospholipase. Alternatively, the 5′-end of the coding sequence may contain a signal peptide coding region that is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the variant phospholipase. However, any signal peptide coding region that directs the expressed polypeptide into the secretory pathway of a host cell of choice may be used in the present invention.

Effective signal peptide coding sequences for filamentous fungal host cells are the signal peptide coding sequences obtained from the genes for Aspergillus oryzae TAKA amylase, Aspergillus niger neutral amylase, Aspergillus niger glucoamylase, Rhizomucor miehei aspartic proteinase, Humicola insolens cellulase, Humicola insolens endoglucanase V, and Humicola lanuginosa lipase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding region that codes for an amino acid sequence positioned at the amino terminus of a variant phospholipase. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region. It may also be desirable to add regulatory sequences that allow the regulation of the expression of the variant phospholipase relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the variant phospholipase would be operably linked with the regulatory sequence.

### Expression Vectors

The various nucleotide and control sequences described above may be joined together to produce a recombinant expression vector that may include one or more (several) convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about the expression of the polynucleotide. The choice of the vector will typically depend on the compatibility of the vector with the host cell into which the vector is to be introduced. The vectors may be linear or closed circular plasmids.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vectors of the present invention preferably contain one or more (several) selectable markers that permit easy selection of transformed, transfected, transduced, or the like cells. A selectable marker is a gene the product of which provides for biocide or viral resistance, resistance to heavy metals, prototrophy to auxotrophs, and the like.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” is defined herein as a nucleotide sequence that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into the host cell to increase production of a phospholipase variant. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra) to obtain substantially pure phospholipase variants.

### Host Cells

A vector comprising a polynucleotide of the present invention is introduced into a host cell so that the vector is maintained as a chromosomal integrant or as a self-replicating extra-chromosomal vector as described earlier. The choice of a host cell will to a large extent depend upon the gene encoding the polypeptide and its source.

The host cell may be any cell useful in the recombinant production of a variant phospholipase, e.g., a prokaryote or a eukaryote.

The prokaryotic host cell may be any Gram positive bacterium or a Gram negative bacterium. Gram positive bacteria include, but not limited to, Bacillus, Streptococcus, Streptomyces, Staphylococcus, Enterococcus, Lactobacillus, Lactococcus, Clostridium, Geobacillus, and Oceanobacillus. Gram negative bacteria include, but not limited to, E. coli, Pseudomonas, Salmonella, Campylobacter, Helicobacter, Flavobacterium, Fusobacterium, Ilyobacter, Neisseria, and Ureaplasma.

The bacterial host cell may be any Bacillus cell. Bacillus cells useful in the practice of the present invention include, but are not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

In one aspect, the bacterial host cell is a Bacillus amyloliquefaciens, Bacillus lentus, Bacillus licheniformis, Bacillus stearothermophilus or Bacillus subtilis cell. In another aspect, the bacterial host cell is a Bacillus amyloliquefaciens cell. In another aspect, the bacterial host cell is a Bacillus clausii cell. In another aspect, the bacterial host cell is a Bacillus licheniformis cell. In another aspect, the bacterial host cell is a Bacillus subtilis cell.

The bacterial host cell may also be any Streptococcus cell. cells useful in the practice of the present invention include, but are not limited to, Streptococcus equisimilis, Streptococcus pyogenes, Streptococcus uberis, and Streptococcus equi subsp. Zooepidemicus cells.

In one aspect, the bacterial host cell is a Streptococcus equisimilis cell. In another aspect, the bacterial host cell is a Streptococcus pyogenes cell. In another aspect, the bacterial host cell is a Streptococcus uberis cell. In another aspect, the bacterial host cell is a Streptococcus equi subsp. Zooepidemicus cell.

The bacterial host cell may also be any Streptomyces cell. Streptomyces cells useful in the practice of the present invention include, but are not limited to, Streptomyces achromogenes, Streptomyces avermitilis, Streptomyces coelicolor, Streptomyces griseus, and Streptomyces lividans cells.

In one aspect, the bacterial host cell is a Streptomyces achromogenes cell. In another aspect, the bacterial host cell is a Streptomyces avermitilis cell. In another aspect, the bacterial host cell is a Streptomyces coelicolor cell. In another aspect, the bacterial host cell is a Streptomyces griseus cell. In another aspect, the bacterial host cell is a Streptomyces lividans cell.

The introduction of DNA into a Bacillus cell may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). The introduction of DNA into an E coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios. 68: 189-2070, by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

In one aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In another aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S .M., and Davenport, R .R., eds, Soc. App. Bacteriol. Symposium Series No. 9, 1980).

In another aspect, the yeast host cell is a Candida, Hansenula, Kluyveromyces, Pichia, Saccharomyces, Schizosaccharomyces, or Yarrowia cell.

In another aspect, the yeast host cell is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis cell. In another aspect, the yeast host cell is a Kluyveromyces lactis cell. In another aspect, the yeast host cell is a Yarrowia lipolytica cell.

In another aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In another aspect, the filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In another aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Methods of Production

In the production methods of the phospholipase variants, the host cells are cultivated in a nutrient medium suitable for production of the phospholipase variant using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

In an alternative aspect, the phospholipase variant is not recovered, but rather a host cell of the present invention expressing a variant is used as a source of the variant.

The phospholipase variant may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein in the Examples.

The resulting phospholipase variant may be recovered by methods known in the art. For example, the polypeptide may be recovered from the nutrient medium by conventional procedures including, but not limited to, collection, centrifugation, filtration, extraction, spray-drying, evaporation, or precipitation.

A phospholipase variant of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure phospholipase variants.

### Methods for Preparing Cakes

A cake maybe prepared in any suitable manner, including, for example, as described in WO 2008/025674, which is hereby incorporated by reference.

The phospholipase may be applied to the cake batter or cake batter ingredients in any suitable manner. The phospholipase may be applied, for example, by direct addition of the phospholipase to a cake batter, by addition of a phospholipase to ingredients used to prepare a cake batter (prior to formation of the cake batter), or by treatment of a phospholipid source, such as, egg yolk lecithin, a non-egg phospholipid or a non-egg protein source separate from the cake batter, followed by inclusion of the treated protein in a cake batter. In one embodiment, the phospholipase is added directly to a cake batter. In another embodiment, the invention provides a method for preparing a cake, comprising:

a) preparing a cake batter comprising egg yolk lecithin;

b) adding a phospholipase variant of the present invention to the cake batter;

c) baking the cake batter to make a cake.

The phospholipase may also be separately added to ingredients used to prepare a cake batter and then the ingredients and the phospholipase may be included in a cake batter. Accordingly, in one embodiment, the present invention provides a method for preparing a cake comprising:

a) providing one or more cake batter ingredients and a phospholipase of the present invention;

b) preparing a cake batter using the composition of a);

c) baking the cake batter to make a cake.

The phospholipase may also be used to prepare a dry mix suitable to prepare a cake batter. In one embodiment, the dry mix includes one or more of the following ingredients: flour, gluten, starch, sweeteners (dextrose, maltose, fructose, lactose, brown and invert sugars, alone or in combination), flavorants, colorants, fat containing components, bulking agents, protein sources, leavening agents, emulsifiers, and/or one or more additional enzymes. The dry mix comprising the phospholipase of the present invention may be added to a cake batter or other cake batter ingredients, as described above.

The present invention also relates to a dry mix composition for producing a cake, comprising a phospholipase variant of the present invention and one or more cake batter ingredients, such as, for example, one or more of the following ingredients flour, gluten, starch, sweeteners (dextrose, maltose, fructose, lactose, brown and invert sugars, alone or in combination), flavorants, colorants, fat containing components, bulking agents, protein sources, leavening agents, emulsifiers and/or one or more additional enzymes.

The method of preparing a cake may further optionally comprise adding a non-egg phospholipid or protein source to the cake batter in combination with a phospholipase of the present invention. As used herein, “non-egg phospholipid or protein source” means a phospholipid or protein source which is not derived from an egg, such as, egg yolk lecithin. Examples of non-egg phospholipid or protein sources include, for example, wheat proteins, casein, whey protein, wheat gluten, legume protein (e.g., soy bean, pea or lupine). The non-egg phospholipid or protein source is preferably added in amounts which are not normally present in a cake recipe. In one embodiment, the present invention provides a method of preparing a cake, comprising:

a) preparing a cake batter comprising egg yolk lecithin,

b) adding a phospholipase variant of the present invention to the cake batter in combination with a non-egg phospholipid or protein source,

c) baking the cake batter to make the cake.

In an embodiment, a non-egg phospholipid or protein source may be treated with a phospholipase of the present invention prior to inclusion in a cake batter. Accordingly, one embodiment comprises a method preparing a cake, comprising:

a) treating a non-egg phospholipid or protein source with a phospholipase variant of the present invention;

b) including the treated non-egg phospholipid source in a cake batter; and

c) baking the cake batter to make the cake.

In an embodiment, the batter used to prepare the cake may contain 0.5-6.0% by weight of non-egg protein, such as, 0.1-6% by weight, such as 0.5-3.0% by weight of non-egg protein, such as, 0.5-2% by weight of non-egg protein.

In an embodiment, the batter may contain 0.1-1.5% by weight of egg lecithin or 5-25% by weight of whole egg (such as, 7-20% or 8-15% by weight of whole egg). The batter may contain 0.1-1.5%, such as, 0.1-1.2%, 0.1-0.9%, 0.2-1.5%, 0.2-0.9%, 0.3-1.5%, 0.3-1.2%, 0.3-0.9% by weight of egg lecithin or 5-25% by weight of whole eggs.

In another embodiment, the amount of egg protein may be reduced in the recipe. For example, the amount of egg protein present in a cake recipe may be from 0.6% to 3% by weight of the cake batter. In another embodiment, the amount of egg protein may be reduced by at least 1-5%, at least 10%, at least 20%, at least 30%, at least 40%, at least 50%, at least 60%, at least 70%, at least 80%, at least 90%, or at least 95%. In an embodiment, the invention also provides a method for preparing a cake using a reduced amount of egg or egg protein or egg yolk lecithin as compared to the amount of egg or egg protein used in conventional cake recipes. In an embodiment, the method further comprises contacting the cake batter with a phospholipase of the present invention, wherein the cake batter contains 0.1-1.5% by weight of egg lecithin or 5-25% by weight of whole egg.

In yet another embodiment, the method further comprises contacting the cake batter with a combination of a non-egg phospholipid or protein and a phospholipase of the present invention, while also using a reduced amount of egg or egg protein in the cake recipe. In another embodiment, the method further comprises contacting the cake batter with a combination of a non-egg phospholipid or protein and a phospholipase of the present invention, wherein the cake batter contains 0.1-1.5% by weight of egg lecithin or 5-25% by weight of whole egg.

The phospholipase variants are added to the batter in an amount effective to obtain a beneficial impact in the batter or cake prepared from the batter, such as, improved properties of volume, texture, such as, cohesiveness, springiness, and elasticity of the cake, or to compensate for reduced egg usage. Non-limiting examples of amounts of phospholipase for use in the present invention include 750 to 2500 LU/kg flour.

The treatment of the phospholipase can occur under normal cake preparation conditions (holding and baking time and temperatures), as are known in the art.

Additional enzymes may be used in combination with the phospholipase in preparing the cake and include, for example, an anti-staling amylase, such as, a maltogenic alpha-amylase (e.g., NOVAMYL from Novozymes A/S), a fungal or bacterial alpha-amylase (e.g., from Aspergillus or Bacillus), amyloglucosidase, a beta-amylase, a xylanase, a protease, a cyclodextrin glucanotransferase or a branching enzyme, a peptidase, a transglutaminase, a lipase, a phospholipase, a cellulase, a hemicellulase, a xylanase, a protein disulfide isomerase, an oxidoreductase, e.g., a peroxidase, a laccase, a glucose oxidase, a pyranose oxidase, a lipoxygenase, or a carbohydrate oxidase.

The present invention also provides a cake batter composition comprising at least one cake batter ingredient and a variant phospholipase, wherein the phospholipase comprises an alteration at one or more positions corresponding to positions 1, 6, 30, 31, 33, 38, 39, 42, 43, 44, 45, 47, 52, 59, 61, 64, 65, 77, 84, 102, 106, 110, 116, 119 or 120 of the mature polypeptide of SEQ ID NO: 2 and/or an amino acid extension at the N and/or C terminus of the phospholipase.

The present invention is further described by the following examples that should not be construed as limiting the scope of the invention.

### EXAMPLES

### Example 1

Phospholipase variants were prepared by altering the nucleic acid sequence (SEQ ID NO:1) encoding the Tuber borchii phospholipase A2 (SEQ ID NO:2). The variants were prepared by saturation mutagenesis. The following phospholipase variants were made:

1. W119*

2. W119G, L120DDATPG

3. L120Q

4. L12OR

5. L1201

6. R61C

7. R61F

8. R61Y

9. R61A

10. R61V

11. R61K

12. R61L

13. R61N

14. R61E

15. R611

16. R61S

17. S116A

18. S116L

19. S116T

20. S116R

21. S116I

22. S116Y

23. S116Q

24. S116P

25. S116H

26. S116F

27. W119T

28. W119*

29. W119P

30. W119G

31. W119A

32. W119K

33. W119N

34. W119V

35. W119H

36. L120E

37. L120S

38. L120A

39. W119H, L1201ATRA

40. W119F, L1201CNSSL

41. W119R

42. W119H, L120CNSSLR

43. W119H, L1201VTRA

44. W119L

45. W119P, L120LCNSSL

46. L120K

47. L120H

48. L120Y

49. L120P

50. L120T

51. L120V

52. S33D

53. S33E

54. D31E

55. K65E

56. S38D

57. S38A

58. N39K

59. Y110F

60. I106V

61. I106P

62. A45F

63. A45V

64. A45D

65. F47Y, A102E

66. A102E

67. F47Y

68. K64R, S1160

69. K64R, W119G, L120DDATPG

70. I106A

71. D84C

72. S52C, D84C

73. S52C

74. G59C

75. G59C, 1770

76. L300

77. S1C, L300

78. L6C, L300

79. L300

80. S38T

81. I106L

82. D42V, R43W

83. W1191

84. W119E

85. W119Q

86. N39C

87. N391

88. N39F

89. A45L

90. P44L, F47L

91. F47W

92. W1190

93. W119S

94. W119F

95. S33M

96. S33C

97. S33G

98. S33W

99. S33A

100. S33Y

101. S33R

102. S33L

103. S33Q

104. N39L

105. N39M

106. N39S

107. N391

108. N39P

109. N39T

110. N39W

111. N39R

112. N39Q

113. A45K

114. A45T

115. A45G

116. A45R

117. A45E

118. A45C

119. F47R

120. F47V

121. F47G

122. F47L

123. F47C

124. A102G

125. A102H

126. A102S

127. S33D, W119G

128. S33D, N39K, W119G

129. S33D, N39K, W119N

130. D31Y, S33D, N39K, W119N

131. N39K, W119G

132. N39K, S116T

133. S1C, L30C, S116T

134. S33D, S116T

135. S33D, N39K

136. S33L, S116T

137. A45C, S116T

138. S1C, L30C, S33Q

139. S1C, L30C, S33L

140. S1C, L30C, S33D

141. S33D, A45C, S116T

142. S33D, A45C

143. SIC, L30C, A45C, S116T

144. SIC, L30C, N39K

### Example 2

Although batter cakes may be made according to any recipe of preference, this experiment addressed the effect of the phospholipase variants in reduced egg cakes. The recipe for the cakes used a high ratio batter cake based on sugar, wheat flour, refined vegetable oil, modified starch, whey powder, baking powder: sodium bicarbonate (E500ii)—sodium acid pyrophosphate (E450i), wheat gluten, salt, emulsifier: sodium stearoyl-2-lactylate (E481) - mono and diglycerides of fatty acids (E471)—lactic acid esters of mono and diglycerides of fatty acids (E472b), stabilizer: carboxymethylcellulose (E466)—guar gum (E412). The sugar is added to 90% when 100% is defined as the sum of flour and starch. The recipe used the commercial cake mix Tegral Satin Crème Cake from Puratos NV/SA, Groot-Bijgaarden, Belgium. The base recipe for a control cake was the following:

| | |
|---|---|
| Tegral Satin Creme Cake mix | 1.000 kg |
| Pasteurized whole egg | 0.350 kg |
| Water | 0.225 kg |
| Rape seed oil | 0.300 kg |

All ingredients were scaled into a mixing bowl and mixed using an industrial mixer (e.g. Bjorn/Bear AR 5 A Varimixer®) with a K-paddle for 2 minutes slow and 2 minutes fast. Scrape down batter from the sides of the bowl in between. Scale 300 grams into aluminum tins (7×19 cm). The cakes are baked in a suitable oven (e.g. Sveba Dahlin deck oven) for 45 min. at 180° C. In order to assess the effectiveness of the phospholipases in reduced egg cakes, fifty percent (50%) egg (175 g) is then replaced with:

A. 42 g wheat gluten, 133 g water and a phospholipase in an amount of 2 kLEU/kg mix;

B. 42 g wheat gluten, 133 g water and a phospholipase in an amount of 5 kLEU/kg mix

C. 42 g wheat gluten, 133 g water and a phospholipase in an amount of 6 KLEU/kg mix.

After cooling the cakes are stored in sealed plastic bags with nitrogen atmosphere at ambient temperature.

### Measuring Volume

Specific volume was calculated from the volume of two cakes without tins divided by the mass of the same cakes measured by rape seed displacement. The unit for specific volume is millilitre per gram.

### Measuring Texture

Texture of the cakes were evaluated on day 1, 7 and 14 after baking, two cakes were used at each occasion, and three slices of cakes were analyzed from each cake. The cohesiveness, springiness, and resilience of the cakes were evaluated using the texture profile analysis (TPA) with TA-XTplus texture analyzer. The Texture profile analysis (TPA) was performed as described in Bourne M. C. (2002) 2. ed., Food Texture and Viscosity: Concept and Measurement. Academic Press. With a circular probe with 491 mm2 area.

Variants with Improved Cake Volume

A number of mutations were made to improve the volume of a cake reduced in egg content.

TABLE 1
Specific volume (ml/g) of cakes with 50% reduced egg relative to a
control cake with standard egg content (i.e., no egg content reduction)
and no phospholipase addition, and which control was assessed as 1.000.
Wt (Tuber albidum phospholipase) was only included in 5 kLEU/kg mix

| | 2 kLEU/kg | 5 kLEU/kg | 6 kLEU/kg |
|---|---|---|---|
| | mix | mix | mix |
| W119A | 0.899 | 0.896 | 0.899 |
| W119V | 0.906 | 0.890 | 0.901 |
| W119T | 0.910 | 0.916 | 0.907 |
| W119H | 0.868 | 0.896 | 0.909 |
| W119R | 0.898 | 0.917 | 0.910 |
| F47Y, A102E | 0.951 | 0.940 | 0.911 |
| F47Y | 0.929 | 0.929 | 0.923 |
| W119K | 0.925 | 0.920 | 0.924 |
| D31Y, S33D, N39K, W119N | 0.940 | 0.955 | 0.928 |
| A45R | 0.936 | 0.940 | 0.938 |
| A45D | 0.966 | 0.936 | 0.938 |
| W119N | 0.939 | 0.945 | 0.948 |
| A45C | 0.935 | 0.951 | 0.951 |
| W119G | 0.926 | 0.946 | 0.952 |
| W119C | 0.970 | 0.976 | 0.953 |
| W119L | 0.951 | 0.981 | 0.955 |
| W119S | 0.975 | 0.943 | 0.956 |
| A45E | 0.961 | 0.963 | 0.956 |
| A45G | 0.934 | 0.988 | 0.956 |
| S33D, N39K, W119G | 0.954 | 0.958 | 0.957 |
| S33Y | 0.962 | 0.997 | 0.960 |
| A102H | 0.948 | 0.946 | 0.963 |
| A102G | 0.942 | 0.973 | 0.963 |
| S33D, W119G | 1.002 | 1.006 | 0.964 |
| W119E | 0.954 | 0.929 | 0.965 |
| S116Q | 0.945 | 0.954 | 0.966 |
| A102S | 0.932 | 0.989 | 0.966 |
| W119Q | 0.961 | 0.961 | 0.968 |
| S33Q | 0.943 | 0.961 | 0.972 |
| N39S | 1.018 | 0.995 | 0.974 |
| I106A | 0.964 | 0.984 | 0.976 |
| N39R | 0.942 | 0.973 | 0.977 |
| S33D | 0.957 | 0.992 | 0.980 |
| N39Q | 0.939 | 0.957 | 0.982 |
| S1C, L30C | 0.951 | 0.971 | 0.983 |
| S33A | 0.989 | 0.998 | 0.984 |
| W119I | 0.979 | 0.989 | 0.984 |
| S116H | 0.967 | 0.994 | 0.984 |
| A45K | 0.958 | 0.982 | 0.985 |
| S116R | 0.966 | 1.001 | 0.988 |
| S33D, N39K, W119N | 0.987 | 0.980 | 0.992 |
| S33C | 0.982 | 0.975 | 0.993 |
| S33W | 0.975 | 0.986 | 0.993 |
| Y110F | 0.942 | 0.928 | 0.997 |
| wt | | 1.000 | |
| S116T | 0.966 | 0.999 | 1.000 |
| N39K | 0.980 | 0.986 | 1.004 |
| S33R | 0.981 | 1.001 | 1.005 |
| S33M | 0.975 | 0.986 | 1.007 |
| I106V | 0.941 | 0.973 | 1.007 |
| N39K, W119G | 0.980 | 0.998 | 1.024 |
| W119F | 0.970 | 0.981 | 1.025 |
| S33L | 0.966 | 0.996 | 1.026 |

Variants with Different Improvements to Cake Cohesiveness

A number of mutations can be made to improve the cohesiveness of a cake reduced in egg content.

TABLE 2
Cohesiveness of cakes with 50% reduced egg relative to a control
cake with standard egg content measured 1, 7, and 14 days after
baking. Cakes are compared to controls stored equal number
of days. Enzyme dose is 5 kLEU/kg mix.

| | Day 1 | Day 7 | Day 14 |
|---|---|---|---|
| D31Y, S33D, N39K, W119N | 0.887 | 0.879 | 0.843 |
| W119T | 0.828 | 0.843 | 0.852 |
| S33D, N39K, W119N | 0.852 | 0.865 | 0.863 |
| N39K, W119G | 0.883 | 0.870 | 0.870 |
| W119K | 0.834 | 0.903 | 0.883 |
| S33D, W119G | 0.875 | 0.856 | 0.884 |
| A45R | 0.861 | 0.882 | 0.889 |
| A45E | 0.868 | 0.952 | 0.890 |
| A45D | 0.870 | 0.870 | 0.891 |
| W119R | 0.835 | 0.881 | 0.891 |
| W119E | 0.892 | 0.913 | 0.891 |
| W119H | 0.848 | 0.883 | 0.892 |
| I106A | 0.874 | 0.906 | 0.895 |
| F47Y, A102E | 0.877 | 0.885 | 0.897 |
| W119A | 0.833 | 0.851 | 0.897 |
| F47Y | 0.875 | 0.879 | 0.901 |
| W119V | 0.867 | 0.859 | 0.901 |
| W119C | 0.883 | 0.949 | 0.908 |
| S116Q | 0.850 | 0.892 | 0.910 |
| A102H | 0.856 | 0.884 | 0.919 |
| A45C | 0.882 | 0.873 | 0.920 |
| A45K | 0.873 | 0.883 | 0.921 |
| N39Q | 0.897 | 0.900 | 0.922 |
| I106V | 0.891 | 0.944 | 0.928 |
| S33Q | 0.606 | 0.958 | 0.934 |
| S116H | 0.873 | 0.918 | 0.934 |
| N39S | 0.876 | 0.928 | 0.936 |
| S116R | 0.858 | 0.925 | 0.938 |
| W119N | 0.859 | 0.937 | 0.941 |
| W119G | 0.854 | 0.897 | 0.941 |
| W119F | 0.884 | 0.921 | 0.941 |
| W119Q | 0.886 | 0.917 | 0.942 |
| S33D, N39K, W119G | 0.893 | 0.898 | 0.942 |
| W119S | 0.896 | 0.967 | 0.945 |
| W119I | 0.889 | 0.938 | 0.946 |
| A102S | 0.895 | 0.934 | 0.951 |
| Y110F | 0.905 | 0.937 | 0.952 |
| W119L | 0.878 | 0.926 | 0.960 |
| S116T | 0.903 | 0.961 | 0.963 |
| S33D | 0.904 | 0.960 | 0.965 |
| S33R | 0.909 | 0.977 | 0.966 |
| S33C | 0.906 | 0.952 | 0.968 |
| S33W | 0.885 | 0.958 | 0.971 |
| N39R | 0.901 | 0.948 | 0.972 |
| S33M | 0.893 | 0.930 | 0.973 |
| N39K | 0.909 | 0.974 | 0.978 |
| S33Y | 0.941 | 0.973 | 0.978 |
| A45G | 0.892 | 0.982 | 0.983 |
| A102G | 0.905 | 0.954 | 0.985 |
| S33L | 0.918 | 0.963 | 0.986 |
| wt | 0.906 | 0.963 | 0.989 |
| control | 1.000 | 1.000 | 1.000 |
| S33A | 0.967 | 0.987 | 1.001 |
| S1C, L30C | 0.896 | 0.947 | 1.005 |

Variants with Different Improvements to Cake Springiness

A number of mutations can be made to improve the springiness of a cake reduced in egg content.

TABLE 3
Springiness of cakes with 50% reduced egg relative to a control cake
with standard egg content measured 1, 7, and 14 days after baking.
Cakes are compared to controls stored equal number
of days. Enzyme dose is 5 kLEU/kg mix.

| | Day 1 | Day 7 | Day 14 |
|---|---|---|---|
| S1C, L30C | 0.916 | 0.914 | 0.898 |
| W119S | 0.941 | 0.926 | 0.899 |
| N39R | 0.882 | 0.911 | 0.911 |
| A102G | 0.893 | 0.919 | 0.914 |
| S116H | 0.909 | 0.904 | 0.918 |
| S116Q | 0.924 | 0.914 | 0.918 |
| S33L | 0.883 | 0.916 | 0.918 |
| S33D | 0.892 | 0.922 | 0.918 |
| S33M | 0.885 | 0.910 | 0.918 |
| W119H | 0.913 | 0.956 | 0.919 |
| S33A | 0.814 | 0.922 | 0.921 |
| W119I | 0.916 | 0.904 | 0.921 |
| S116R | 0.919 | 0.908 | 0.922 |
| S116T | 0.896 | 0.921 | 0.922 |
| wt | 0.906 | 0.930 | 0.922 |
| A45C | 0.892 | 0.934 | 0.925 |
| N39K | 0.908 | 0.918 | 0.927 |
| I106A | 0.932 | 0.934 | 0.927 |
| F47Y, A102E | 0.918 | 0.918 | 0.928 |
| W119K | 0.963 | 0.895 | 0.928 |
| A45G | 0.918 | 0.909 | 0.929 |
| W119N | 0.923 | 0.915 | 0.930 |
| S33Y | 0.898 | 0.920 | 0.931 |
| W119A | 0.921 | 0.936 | 0.931 |
| F47Y | 0.942 | 0.922 | 0.931 |
| W119V | 0.921 | 0.944 | 0.933 |
| W119R | 0.960 | 0.929 | 0.933 |
| Y110F | 0.894 | 0.932 | 0.934 |
| S33C | 0.901 | 0.933 | 0.935 |
| S33W | 0.902 | 0.931 | 0.935 |
| I106V | 0.922 | 0.932 | 0.936 |
| S33D, N39K, W119G | 0.932 | 0.942 | 0.937 |
| W119L | 0.912 | 0.916 | 0.937 |
| A102S | 0.934 | 0.948 | 0.938 |
| A102H | 0.934 | 0.953 | 0.939 |
| A45E | 0.906 | 0.929 | 0.942 |
| W119F | 1.009 | 0.932 | 0.943 |
| A45D | 0.926 | 0.922 | 0.945 |
| W119G | 0.922 | 0.936 | 0.946 |
| W119T | 0.921 | 0.934 | 0.946 |
| N39S | 0.975 | 0.935 | 0.947 |
| W119E | 0.907 | 0.895 | 0.947 |
| W119Q | 0.920 | 0.957 | 0.949 |
| A45K | 0.926 | 0.951 | 0.950 |
| S33Q | 0.590 | 0.922 | 0.950 |
| S33D, N39K, W119N | 0.920 | 0.940 | 0.950 |
| S33R | 0.893 | 0.919 | 0.951 |
| S33D, W119G | 0.928 | 0.940 | 0.952 |
| N39K, W119G | 0.888 | 0.937 | 0.953 |
| N39Q | 0.763 | 0.957 | 0.958 |
| A45R | 0.923 | 0.946 | 0.959 |
| W119C | 0.958 | 0.915 | 0.963 |
| D31Y, S33D, N39K, W119N | 0.977 | 0.957 | 0.995 |
| Control | 1.000 | 1.000 | 1.000 |

Variants with Different Improvements to Cake Resiliency

A number of mutations can be made to improve the resiliency of a cake reduced in egg content.

TABLE 4
Resiliency of cakes with 50% reduced egg relative to a control cake
with standard egg content measured 1, 7, and 14 days after baking.
Cakes are compared to controls stored equal number
of days. Enzyme dose is 5 kLEU/kg mix.

| | Day 1 | Day 7 | Day 14 |
|---|---|---|---|
| D31Y, S33D, N39K, W119N | 1.062 | 0.835 | 0.737 |
| S33D, N39K, W119N | 0.713 | 0.775 | 0.762 |
| W119T | 0.681 | 0.756 | 0.763 |
| N39K, W119G | 0.750 | 0.791 | 0.772 |
| W119H | 0.694 | 0.769 | 0.789 |
| A45R | 0.723 | 0.784 | 0.796 |
| S33D, W119G | 0.757 | 0.769 | 0.807 |
| W119E | 0.784 | 0.851 | 0.811 |
| W119A | 0.684 | 0.770 | 0.819 |
| F47Y, A102E | 0.741 | 0.780 | 0.822 |
| W119K | 0.683 | 0.836 | 0.825 |
| S33Q | 0.510 | 0.868 | 0.830 |
| A45D | 0.748 | 0.785 | 0.838 |
| F47Y | 0.742 | 0.776 | 0.838 |
| N39Q | 0.747 | 0.830 | 0.842 |
| S116H | 0.703 | 0.803 | 0.843 |
| S116Q | 0.673 | 0.786 | 0.843 |
| W119R | 0.688 | 0.799 | 0.845 |
| I106A | 0.748 | 0.825 | 0.850 |
| W119V | 0.732 | 0.784 | 0.853 |
| S116R | 0.685 | 0.838 | 0.863 |
| A45C | 0.759 | 0.783 | 0.866 |
| A45E | 0.751 | 0.875 | 0.870 |
| I106V | 0.771 | 0.873 | 0.873 |
| S116T | 0.756 | 0.868 | 0.873 |
| S33R | 0.775 | 0.907 | 0.876 |
| N39S | 0.760 | 0.872 | 0.876 |
| Y110F | 0.782 | 0.864 | 0.881 |
| A102H | 0.724 | 0.816 | 0.883 |
| W119N | 0.714 | 0.879 | 0.885 |
| A102S | 0.765 | 0.847 | 0.887 |
| N39R | 0.771 | 0.880 | 0.890 |
| S33D | 0.771 | 0.884 | 0.892 |
| S33M | 0.771 | 0.829 | 0.894 |
| W119F | 1.392 | 0.862 | 0.894 |
| S33D, N39K, W119G | 0.774 | 0.799 | 0.895 |
| W119C | 0.784 | 0.943 | 0.896 |
| W119Q | 0.789 | 0.876 | 0.904 |
| N39K | 0.779 | 0.893 | 0.905 |
| S33L | 0.804 | 0.883 | 0.905 |
| S33Y | 0.842 | 0.902 | 0.906 |
| W119G | 0.718 | 0.830 | 0.907 |
| S33C | 0.806 | 0.883 | 0.907 |
| S33A | 0.876 | 0.918 | 0.910 |
| A102G | 0.773 | 0.879 | 0.913 |
| A45G | 0.759 | 0.902 | 0.914 |
| W119S | 0.787 | 0.927 | 0.915 |
| W119I | 0.767 | 0.897 | 0.920 |
| A45K | 0.754 | 0.828 | 0.922 |
| wt | 0.787 | 0.890 | 0.925 |
| W119L | 0.745 | 0.862 | 0.927 |
| S33W | 0.765 | 0.893 | 0.928 |
| S1C, L30C | 0.766 | 0.871 | 0.976 |
| Control | 1.000 | 1.000 | 1.000 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A method for preparing a cake, said method comprising:
a) preparing a cake batter comprising egg yolk lecithin and a variant of a parent phospholipase, wherein the variant phospholipase comprises an alteration at one or more positions corresponding to positions 1, 6, 30, 31, 33, 38, 39, 42, 43, 44, 45, 47, 52, 59, 61, 64, 65, 77, 84, 102, 106, 110, 116, 119 or 120 of the mature polypeptide of SEQ ID NO: 2, wherein the variant phospholipase has phospholipase activity and comprises an amino acid sequence having at least 50% identity with of the mature polypeptide SEQ ID NO:2 or the mature polypeptide of SEQ ID NO:3; and
b) baking the cake batter to make a cake.

2. (canceled)

3. The method of claim 1, wherein the parent phospholipase is obtained from a fungus.

4. The method of claim 1, wherein the parent phospholipase is obtained from genus Tuber.

5. The method of claim 1, wherein the parent phospholipase is a phospholipase obtained from Tuber borchii.

6. The method of claim 1, wherein the parent phospholipase is a phospholipase obtained from Tuber albidum.

7. The method of claim 1, wherein the parent phospholipase comprises an amino acid sequence of the mature polypeptide of SEQ ID NO:2 or the amino acid sequence of the mature polypeptide of SEQ ID NO:3.

8. The method of claim 1, wherein the batter comprises 0.1-1.5% by weight of egg lecithin or 5-25% by weight of whole egg.

9. The method of claim 1, wherein the batter comprises a non-egg protein.

10. The method of claim 9, wherein the batter comprises at least 0.5% by weight of a non-egg protein.

11. The method of claim 9, wherein the batter comprises at least 0.5-6.0% by weight of non-egg protein.

12. The method of claim 9, wherein the non-egg protein is selected from the group consisting of wheat proteins, casein, whey protein, wheat gluten, and legume protein

13. The method of claim 1, wherein the batter comprises a non-egg phospholipid.

14. The method of claim 9, wherein the batter comprises 0.3-1.5% by weight of egg lecithin or 5-25% by weight of whole egg.

15. The method of claim 1, wherein the phospholipase is added in to the cake batter in the form of a dry mix.

16. The method of claim 1, wherein the phospholipase is added to ingredients of a cake batter prior to addition to the cake batter.

17. A cake batter comprising a variant of a parent phospholipase, wherein the variant phospholipase comprises an alteration at one or more positions corresponding to positions 1, 6, 30, 31, 33, 38, 39, 42, 43, 44, 45, 47, 52, 59, 61, 64, 65, 77, 84, 102, 106, 110, 116, 119 or 120 of the mature polypeptide of SEQ ID NO: 2, wherein the variant phospholipase has phospholipase activity and comprises an amino acid sequence having at least 50% identity with of the mature polypeptide SEQ ID NO:2 or the mature polypeptide of SEQ ID NO:3.

18. A cake batter dry mix comprising a cake batter ingredient and a variant of a parent phospholipase, wherein the variant phospholipase comprises an alteration at one or more positions corresponding to positions 1, 6, 30, 31, 33, 38, 39, 42, 43, 44, 45, 47, 52, 59, 61, 64, 65, 77, 84, 102, 106, 110, 116, 119 or 120 of the mature polypeptide of SEQ ID NO: 2, wherein the variant phospholipase has phospholipase activity and comprises an amino acid sequence having at least 50% identity with of the mature polypeptide SEQ ID NO:2 or the mature polypeptide of SEQ ID NO:3.


SEQUENCE LISTING (3 sequences)

SEQ ID NO: 1 - DNA, 832 bp, Tuber albidum
aatggtcaagattgctgccattgtcctcctaatgggaattctagccaatgctgccgccatccctgtcagcgagccagcagccctggcgaagcgtggaaacgctgaggtcattgctgaacaaactggtgatgtcccggatttcaacactcaaattacagagccaactggggagggagaccgtggggatgtggtcgacgaaaccgatttgtccacggatattgtcccagagaccgaggctgcttccttcgccgctagttcagtatctgcagcctcaccagcatctgacaccgacaggcttctctactcaacctccatgcccgccttcttgactgctaagcgcaataagaaccccggcaacttggactggagcgatgatggatgcagcaactccccggacaggcctgcagggtttaacttccttgactcgtaagtcctccttcatttatgctatctacattcactaatattcgaacagctgcaagcgtcacgacttcgggtaccgcaactacaagaagcagcgccgcttcacagagcctaatcgcaagcgcattgatgacaatttcaagaaggacctatataatgagtgcgccaagtactctggcctccaatcctggaaaggtgttgcctgccgcaaaatcgcgaacacttactacgatgctgtacgctccttcggttggttgtaaatgtgcggaagagatatcaagtgggatcgaggaagaggatggtgaaagagctgagaggtggatttctttacattccgcaatggctactacagaagaactgtgctcctcaaatttaatctcatttttgtgtctatctatccactctagaa

SEQ ID NO: 2 - protein, 210 aa, Tuber albidum
MVKIAAIVLLMGILANAAAIPVSEPAALAKRGNAEVIAEQTGDVPDFNTQITEPTGEGDRGDVVDETDLSTDIVPETEAASFAASSVSAASPASDTDRLLYSTSMPAFLTAKRNKNPGNLDWSDDGCSNSPDRPAGFNFLDSCKRHDFGYRNYKKQRRFTEPNRKRIDDNFKKDLYNECAKYSGLQSWKGVACRKIANTYYDAVRSFGWL

SEQ ID NO: 3 - protein, 211 aa, Tuber borchii
MVKIAAIILLMGILANAAAIPVSEPAALNKRGNAEVIAEQTGDVPDFNTQITEPTGEGDRGDVADETNLSTDIVPETEAASFAASSVSAALSPVSDTDRLLYSTAMPAFLTAKRNKNPGNLDWSDDGCSKSPDRPAGFNFLDSCKRHDFGYRNYKKQHRFTEANRKRIDDNFKKDLYNECAKYSGLESWKGVACRKIANTYYDAVRTFGWL